(19) United States
(12) Patent Application Publication
(10) Pub. No.: US 2008/0026476 A1
(43) Pub. Date: Jan. 31, 2008

(54) Device and Method for Detecting Blood Coagulation

(76) Inventors: Steven Howell, Pesthshire (GB); Robert J. Davies, Bedford (GB); David E. Williams, Auckland (NZ)

Correspondence Address: DICKSTEIN SHAPIRO LLP, 1825 EYE STREET NW, Washington, DC, 20006-5403, US

(21) Appl. No.: 11/596,742

(22) PCT Filed: May 20, 2005
(86) PCT No.: PCT/GB05/02017

§ 371 Date: Jul. 17, 2007

(30) Foreign Application Priority Data
May 20, 2004 (GB) 0411281.9
Jul. 7, 2004 (GB) 04115245.0

(51) Int. Cl. G01N 33/86; G01N 21/01; G01N 33/48

(52) U.S. Cl. 436/69; 422/68.1; 422/73; 422/82.11

(57) ABSTRACT

A device is provided for use with a reader for determining coagulation of a sample of biological fluid. The device comprises a structure having an at least one chamber for containing a sample of biological fluid. A coagulation reagent capable of interacting with the fluid sample is provided within the device. The chamber further contains either: a plurality of particles susceptible to movement in a magnetic field; or one particle susceptible to movement in a magnetic field.

The present invention relates to a method of, and a device and system for determining coagulation of a sample of biological fluid.

In particular but not exclusively the invention relates to the determination of prothrombin time in serum, plasma or whole blood.

According to a first aspect of the invention there is provided a method of determining the coagulation status of a sample of biological fluid by interaction with a coagulation reagent, the method comprising, in any order of steps (a)-(c) or simultaneously:
(a) causing the sample of biological fluid to become disposed in a device, the device having a chamber containing a particle which is susceptible to movement in a magnetic field;
(b) successively applying first and second magnetic fields to cause the said particle to move to and fro within the chamber;
(c) optically monitoring the chamber to establish a change in said to and fro movement of said particle and:
(d) correlating the change in particle movement with the coagulation status of the fluid sample.

In one embodiment, the coagulation reagent is disposed in the device prior to step a).

According to a second aspect of the present invention, there is provided a device for use with a reader for determining coagulation of a sample of biological fluid, the device comprising a structure having a chamber for containing a sample of biological fluid, and wherein a coagulation reagent capable of interacting with the fluid sample is provided within the device, the chamber containing a number of particles susceptible to movement in a magnetic field.

Preferably, each particle has a major axis greater than 5 um in length. More preferably, each particle has a major axis between 5 um and 12 um in length. More preferably still, each particle has a major axis that is substantially 10 um in length.

According to a third aspect of the present invention, there is provided a device for use with a reader for determining coagulation of a sample of biological fluid, the device comprising a structure having at least one chamber for containing a sample of biological fluid, and wherein a coagulation reagent capable of interacting with the fluid sample is provided within the device, the at least one chamber containing one particle susceptible to movement in a magnetic field.

Preferably, the particle has a major axis between 300 um and 700 um in length. More preferably, the particle has a major axis between 400 um and 600 um in length. More preferably still, the particle has a major axis that is substantially 500 um in length.

Preferably, the particle has a thickness between 50 um and 100 um. More preferably, the particle has a thickness that is substantially 70 um.

Preferably, the particle is shaped like one of the groups of shapes comprising: a disc, a sphere, a torus, an ellipsoid, and an oblate spheroid.

It is an object of embodiments of the present invention to provide a device wherein at least one particle in a chamber is arranged moves in a to and fro movement when subjected to an appropriate magnetic field.

Embodiments of the present invention are suitable for use with a reader for determining the coagulation status of a sample of biological fluid wherein the reader does not require any moving parts. In such a reader, an optical sensor may be used to monitor the position of the at least one particle. When the biological fluid coagulates, the amplitude of movement of the at least one particle reduces.

Another aspect of embodiments of the present invention is the ratio of the dimensions of the particle with respect to the dimensions of the chamber. Preferably, the chamber is as small as possible to reduce the volume of sample fluid required. In embodiments of the present invention, the chamber has dimensions of 1.6 mm long, 1 mm wide and 125 um high.

In embodiments of the present invention, the axis of to and fro movement of the particle within the chamber is arranged along the length of the particle and along the length of the chamber. In such embodiments, the ratio of the particle length to chamber length is preferably between 0.1 and 0.5. More preferably, this ratio is between 0.2 and 0.4. In such embodiments, the ratio of the particle width to chamber width is preferably between 0.1 and 0.75. Further, in such embodiments, the ratio of the particle height to chamber height is between 0.2 and 0.5.

In embodiments of the present invention, the ratio of the volume of the particle to the volume of the chamber is between 0.1 and 0.5. Preferably, this ratio is 0.42.

According to embodiments of the invention there is provided a device for use with a reader for determining coagulation of a sample of biological fluid, the device comprising a structure having a chamber for containing a sample of biological fluid, and wherein a coagulation reagent capable of interacting with the fluid sample is provided within the device, the chamber containing a particle which is susceptible to movement in a magnetic field.

According to embodiments of the invention there is provided a reader for use with a device according to the second aspect for determining coagulation of a sample of biological fluid the reader comprising:—magnetic means arranged to successively apply first and second magnetic fields to cause a said particle to move to and fro within the chamber; optical monitor means associated with the chamber to establish a change in said to and fro particle movement.

According to a further aspect of the invention there is provided a system for determining coagulation of a sample of biological fluid, comprising a magnetic drive means and structure defining a chamber, the chamber containing a particle capable of moving under the influence of a magnetic field, the magnetic drive means being arranged in use to co-operate with the particle to cause it to move back and forth in the chamber, the device further comprising at least one light detection means having an input disposed to be selectively occluded by the said particle.

According to yet a further aspect, the invention provides for a method of manufacture of a test-strip device.

According to yet a further aspect, the invention provides for a solenoid arrangement.

According to a yet further aspect, the invention provides for a method of measuring a coagulation time of a fluid sample.

The term coagulation as used herein includes time based measurements resulting in the formation of a clot such as prothrombin time, activated partial thromboplastin time, protein C activation time and thrombin time. The device and system embodying the invention may also be used to measure changes in viscosity resulting from fibrin formation and platelet aggregation.

The nature of the reagent used to induce coagulation will depend upon the test to be performed. Such reagents may be chosen from enzymes such as those derived from snake venoms, or thrombin, or other active proteases, surface-active substances, such as silicates or phenol derivatives, activated blood platelets or blood platelet-activating substances, such as thrombin, collagen, adrenalin or adenosin diphosphate, or by the optional addition of coagulation-supporting substances, such as buffering substances, calcium chloride and/or phospholipids.

In an embodiment, a particle is chosen that is not permanently magnetic namely having minimal magnetic remanence and coercivity such that it is able to move back and forth between the two pole pieces of the respective solenoids.

In an embodiment, the device comprises outer upper and lower surfaces which are bound by side-walls in which is provided a fluidic pathway. An embodiment of the test-strip comprises a sample entry port for introduction of the fluid sample, optionally one or more fluid conduits and one or more fluid chambers. The sample entry port, fluid conduits and sample chambers are in fluidic connection such that sample applied to the sample entry port is able to flow along the fluid conduit and into the fluid chamber. A further fluidic conduit may be connected to the fluid outlet port as well as means provided downstream from the fluid outlet port to stop the flow of fluid sample, such as a capillary break. The device is also provided with a vent which serves to vent gases that may be contained within the device and to allow the device to fill with sample fluid. In an embodiment the fluidic dimensions are such that fluid is carried into and/or through the device by capillary action. Controlling the flow of fluid solely by capillary action is preferred as the flow of fluid is independent upon the orientation of the device or the orientation of the fluidic passageways, namely, gravitational forces are insignificant. Alternatively however, the fluid may travel through the device under the influence of forces other than capillary such as by electrokinetic pumping, gravity or a combination of gravity and capillary action etc. A single fluid conduit may connect the sample entry port which then may then bifurcate to supply two fluid chambers or trifurcate to supply three fluid chambers and so on. Alternative, more than one fluid conduit may connect the sample entry port.

Where the test requires the use of a coagulation reagent to promote or retard coagulation of the fluid sample, a coagulation reagent is disposed within the chamber. Alternatively or additionally the coagulation reagent may be provided elsewhere within the device upstream from the fluid chamber. Different tests may be performed within the same device, for example by providing an appropriate coagulation reagent in one test chamber and another reagent in a second test chamber.

In an embodiment, the fluidic arrangement of the test-strip has a housing which may also serve to define the fluidic regions themselves. The material of the test-strip may be any suitable such as glass or a plastics material such as polycarbonate. In an embodiment the material is chosen to be light permeable.

In embodiments, the reader has an external housing as well as magnetic drive means, means by which to engage or receive the device, location means to precisely locate the device within the device, light source and light detection means, processing means for processing a signal received by the light detection means, a power source or means to receive a source of power, display means for providing instructions to the user, for displaying any messages such as error messages and for displaying a result processed by the processing means as well as memory means for storing information. The reader may have on-board heating means which is able to heat the fluid sample and maintain the temperature at a constant value for the duration of the measurement. The result to be displayed by the reader may be expressed in terms of an internationalised normalised ratio or INR. Typically the device is intended to be single use and the reader is designed to be reusable. However as an alternative, the device and reader may be provided as a single disposable element.

The time to coagulation may be defined as the time taken for the particle to cease movement or the time determined by the reader as having ceased movement or has slowed down to such an extent that it is considered as having ceased. An example of where the reader might determine that the particle has ceased movement in whereby the particle no longer continues to travel in a to and fro movement within the chamber but effectively hovers about a point, trying to move in a particular direction but prevented from doing so by the coagulating sample. As an alternative to determining the time to coagulation the device may also be used to determine the change or rate of change in the movement of the particle during the coagulation process. The time determined as to when the sample has coagulated will to some extent be determined by factors such as the magnetic field strength, the residence time of the particle which in turn will be determined by the switching time between the solenoids, as well as the shape, size and weight of the particle which in turn will determine the particle momentum. If the particle momentum is too great, the particle may continue to move even the blood has coagulated to quite a degree. On the other hand, if the particle momentum is too low, the particle may become stopped by a few strands of fibrin or by a small clot. In this respect, the magnetic field strength need not be constant for the duration of the measurement and may vary depending upon for example the speed of the particle and the time of the test.

In embodiments a single magnetically susceptible particle is employed as this has been shown to provide a more absolute cut-off point in determining the onset of coagulation in the sense that the presence of the particle is either detected or not. According to other embodiments, more than one particle may be used. However, it has been found that the use of more than one particle can result in a particle trail occurring as a result of particles moving back and forth through the fluid sample in the chamber. In these circumstances it was found that the determination of the coagulation time was not so absolute. Furthermore, a single particle of an appropriate size advantageously serves to cause bulk mixing which many small particles do not. In addition it was observed that the use of a number of particles having a particle size of the order of 2-12 um has a tendency to move the red-blood cells aside as a consequence of the particles moving back and forth within the chamber.

However, employing a single particle has potential drawbacks. It has to represent what is happening across a significant proportion of the fluid sample. During manufacture, consistent placement of a particle into the chamber is advantageous as well as being able to measure its presence and absence. Therefore in embodiments the particle has been chosen to be rather large both in absolute terms and in terms of the ratio of size of particle to the volume of the chamber. The range of dimensions of the particle may be described in absolute terms and/or may be described as a ratio of particle number to chamber volume, a ratio of particle size to fluid volume or as a ratio of the cross-sectional area of the particle to the effective cross-sectional area of the fluid chamber through which the particle moves. From a microfluidic point of view, a ratio of the cross-sectional area of particle to fluid of or less than about 1/9 creates near-optimum fluid flow.

Where the particle is of a non-uniform shape, the cross-sectional area of the particle will be defined by the maximum cross-sectional area or aspect-ratio of the particle at any point along its length.

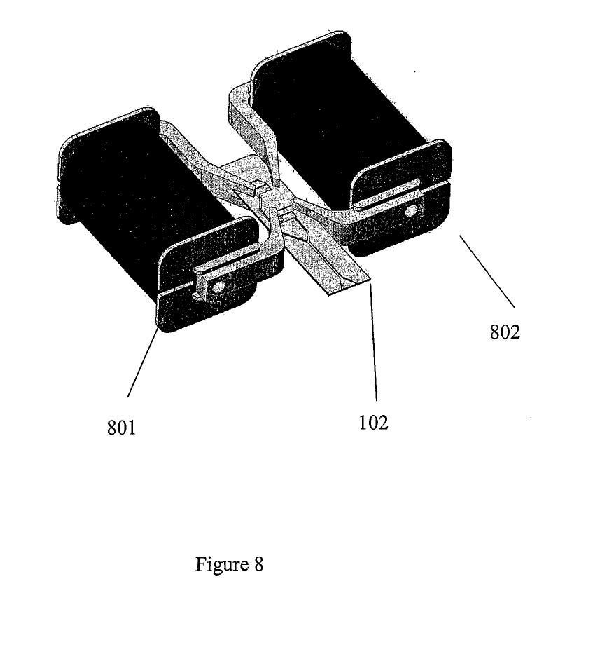

In an exemplary embodiment, the particle used is approximately pancake-shaped and has a diameter of 400-600 um and a thickness of 70 um. The fluid chamber of this embodiment has the dimensions of 175 um in height×1000 um in width and a length of 2000 um which corresponds to a volume of 350 nL and which represents a ratio of the cross-sectional area of the particle to the cross-sectional area through which the particle moves of approximately 1:5. A device having the above chamber dimensions is shown in FIG. 8. In this particular case, there are two chambers and an additional fluid conduit volume of 300 nL, thus requiring a total volume of 1 uL.

In different embodiments the particles have different sizes, shapes and densities and the size of particle chosen will depend upon various factors such as the volume and cross-sectional aspect ratio of the chamber, as well as practical considerations such as ease of manufacture of the device and for quality control purposes to determine whether the particle is indeed present. Ideally the particle should be of a size and/or shape such its travel within the chamber is not impeded or influenced by the fluid inlet or outlet. Other shapes may be contemplated, for example wherein the outer surface of the particle is curved to enable the particle to be resuspended into the fluid sample more effectively. Where more than one particle is used, the individual size and/or shape of the particles may vary and be different compared to the size of the particle where only one is used.

The shape and composition of the particle has been shown to have an effect on the result. Some shapes provide for erratic movement of the particle through the liquid. In the exemplary embodiment discussed above the particle is produced by squashing individual spheres to provide the pancake-shape.

The particle may be provided as individual discs obtained from a sheet of metal for example by punching, cutting, lasering, chemical etching or partial chemical etching followed by cutting. The presence of silica in the iron particle which is believed to reduce magnetic remanence may also make a difference as to the movement properties of the particle.

The particle may be chosen to be porous or non-porous. According to one embodiment the particle may be porous such that the coagulation reagent may be deposited within the particle itself. Alternatively the coagulation reagent may be coated onto the surface of the particle. This has the advantage of avoiding the need to separately dispense coagulation reagent into the chamber.

The chamber may be any convenient shape and its volume ranges typically from about 100 nL to 10 μL. The volume required by the device will depend upon the number of chambers and for a device having two chambers the volume requirement will typically range from about 250 nL-25 μL.

A test strip defining one or more fluid chambers has (in the or each chamber) a single magnetically susceptible particle. In use the particle is caused to move back and forth or to and fro within the chamber under the influence of a magnetic field. The magnetic field is provided by a magnetic drive means, such as a solenoid system comprising two or more solenoids. As an alternative however, the magnetic drive means may comprise a solenoid and a permanent magnet.

In an embodiment, the test-strip has a three-laminae construction with a lower layer, a middle layer and an upper layer. The middle layer serves to define the geometry of the fluid chambers as well as any other fluidic connections and the upper and lower layers serve to define respectively the upper and lower surfaces of the fluid chambers. In an embodiment each fluid chamber is in fluidic connection with an inlet channel for introduction of fluid sample into the fluid chamber and a vent to ensure adequate filling of the chamber.

In embodiments, two sets of optics are provided within the test device per chamber and are located such as to optically interrogate different positions of each chamber, whereby both the presence and absence of the magnetic particle in each position is determined. According to other embodiments a single set of optics is provided to optically interrogate a region of the chamber, for example a middle region of the chamber

The chamber may be designed such that the inlet and outlet ports are diametrically opposed. The particle may initially be positioned towards the inlet or outlet side of the chamber so as to avoid air-bubbles.

The middle lamina defining the fluidic geometry of the test-strip may be fully or partially cut. The venting channel employs a partially cut channel which then becomes a fully cut wider channel at its distal end thus providing an effective capillary break and stops the egress of fluid from the test-strip.

The described embodiment employs two sets of optics per chamber positioned so as to detect the particle at either end of the chamber in an orientation designed to capture the mode of motion of the particle. This has been shown to provide accurate and consistent results. With only one set of optics, it is possible that at the onset of coagulation, the particle can hover in and out of the zone of optical detection creating the illusion that movement is still occurring. With two sets of optics, for example positioned at either ends of the chamber, the presence or absence may be more reliably determined.

Due to the extremely small size of the fluid chamber providing two optical detectors and two LEDS in close proximity to the chamber becomes very difficult. Consequently, in some embodiments fibre optics are employed. In other words, LEDs or other light sources, and optical detectors, such as photodiodes are positioned remotely from the chamber and are optically connected to an optical fibre. The fibres, which are smaller than the light sources or detectors can then be positioned in close proximity to the chamber. In other embodiments light guides other than optical fibres are be employed such as the fluid conduits themselves. In yet other embodiments optics of a sufficiently small size are employed. In some embodiments the light source and light detector are positioned on the same side of a chamber. In these embodiments, in use, light from the light source passes into the chamber and is reflected back towards the light detector. In alternative embodiments the light source and detector are positioned on opposite or alternative sides of the chamber. In yet other embodiments, components that allow the transition of the light source from plastic fibre optic to an airpath are used. As yet a further alternative, die mounted components in a custom optical assembly may be employed.

The optics may also serve to determine the presence or absence of fluid sample in the chamber by determining a change in the fluid characteristics of the chamber. The optics may also serve to determine the time of arrival of fluid into the chamber or time when the chamber has been filled. This information may then be used to signal commencement of the measurement process.

In an embodiment, two chambers are employed to provide a controlled coagulation reaction. One chamber has a coagulation reagent and is used for detection of the coagulation time. The other chamber has a reagent which provides a fixed time of coagulation independent of the blood sample and therefore serves as a control. Alternatively the control reagent may serve to delay the coagulation reaction or ensure that it does not occur.

In one embodiment four solenoids are employed, two per chamber—however this proves to be expensive and heavy.

In another aspect the invention relates to a device for use with an optical reader for determining coagulation of a sample of biological fluid, having a chamber for containing a said sample and a channel for admitting said biological fluid into said chamber, wherein the channel and the chamber together have a volume of less than 3 μL.

In an embodiment, the device has a volume less than 1 μL.

In an embodiment, the device has a volume less than 250 nL

In an embodiment, the device has a volume of substantially 100 nL.

An embodiment has integral means for penetrating the skin, the said means defining a conduit which forms at least part of said channel.

In yet other aspects of the invention there are provided a device for use with a reader, and to a device having at least one particle susceptible of movement, having a chamber for containing a sample and a channel for admitting biological fluid into said chamber, wherein the channel and the chamber together have a volume of less than 3 μL. The reader may be optical.

Exemplary embodiments of the invention will now be described with reference to the accompanying drawings, in which:

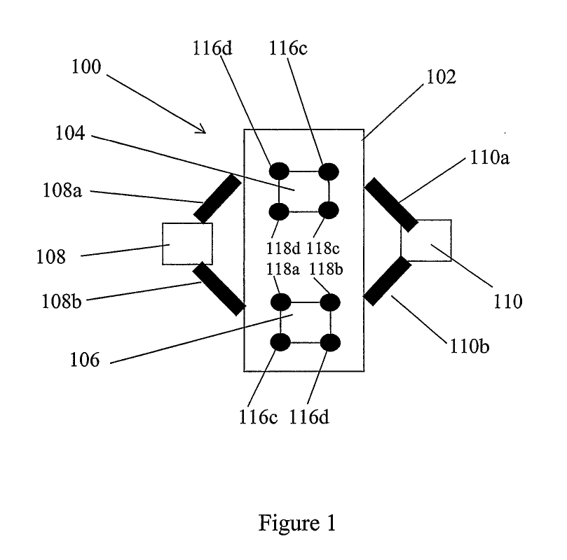

FIG. 1 shows a schematic overview of a device embodying the present invention;

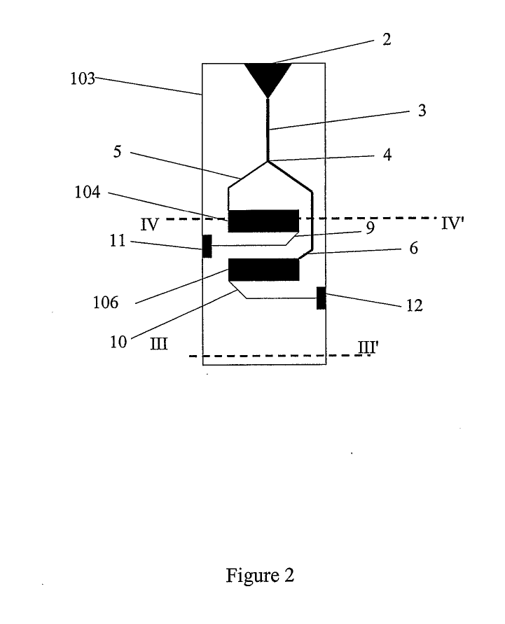

FIG. 2 shows a schematic in plan view of one of the laminae of the test strip of FIG. 1;

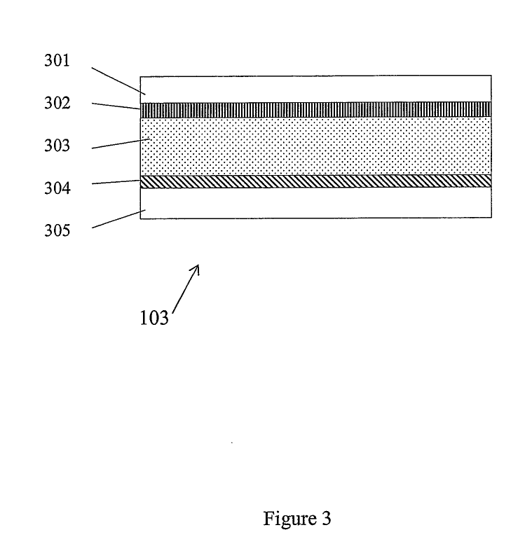

FIG. 3 shows a partial cross-section along line III-III′ of FIG. 2;

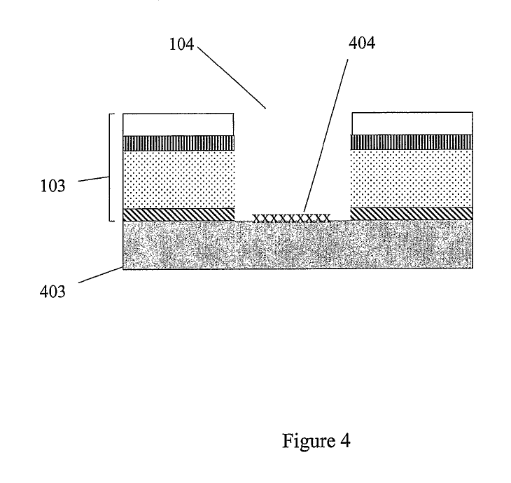

FIG. 4 shows a cross-section along line IV-IV′ of FIG. 2 with a lower lamina in position;

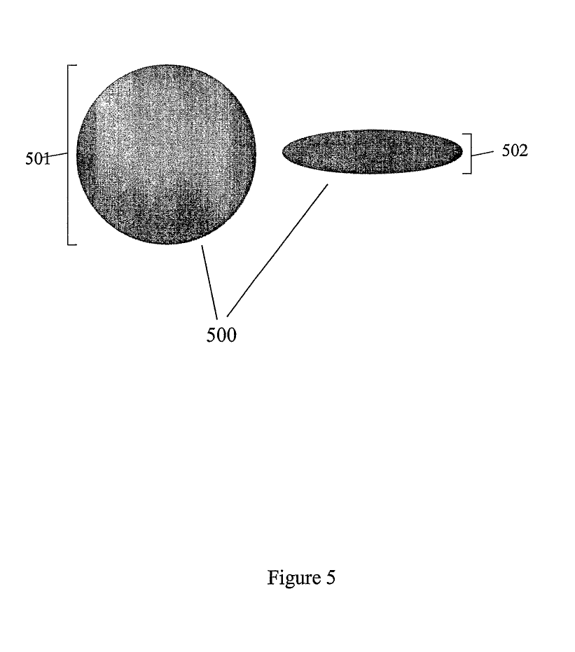

FIG. 5 shows a schematic diagram of exemplary magnetic particles for use with the invention;

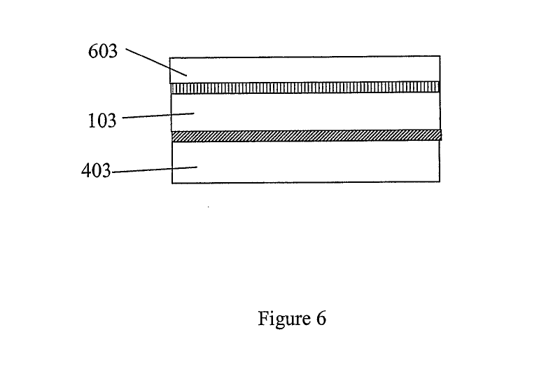

FIG. 6 shows a cross-section along line III-III′ of FIG. 2;

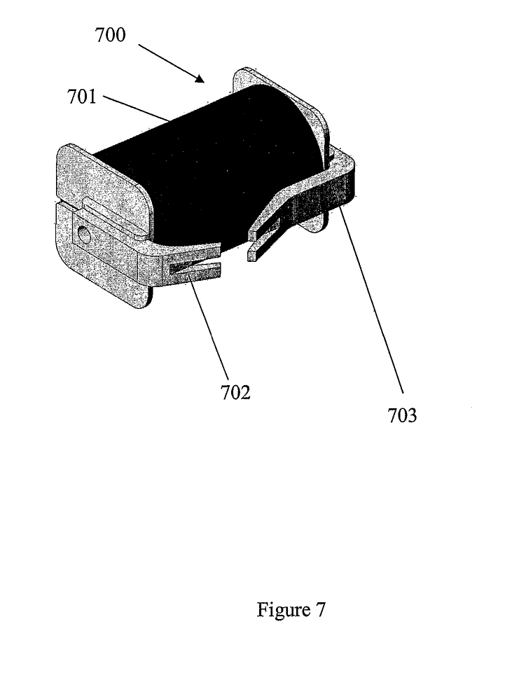

FIG. 7 shows a perspective view of an exemplary solenoid for use in the invention;

FIG. 8 shows a perspective view of a test strip assembled with two solenoids;

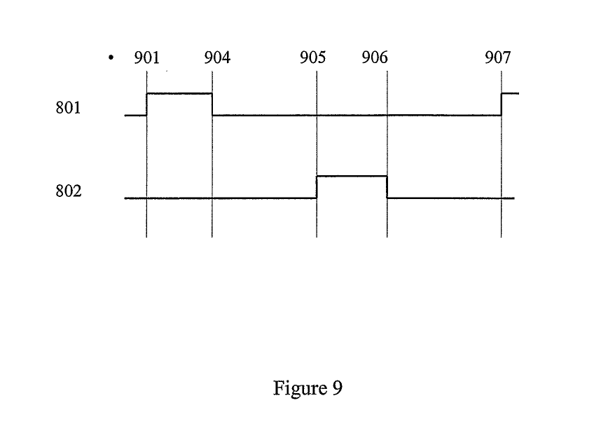

FIG. 9 shows a timing diagram of solenoid operation;

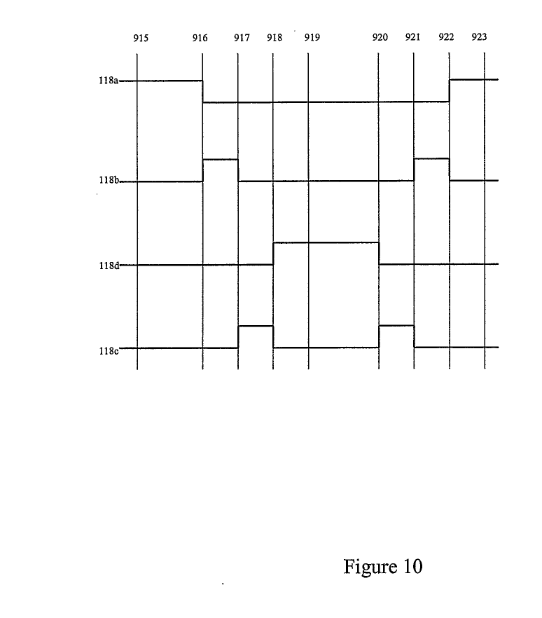

FIG. 10 shows a timing diagram of light emission and detection; and

FIG. 11 shows a graph illustrating detection of a clotting event.

FIG. 1 shows an exemplary embodiment of a system (100) for determining coagulation of a sample of biological fluid, formed of a test strip (102) and a solenoid arrangement (108, 110). As shown the test strip has two generally rectangular chambers (104, 106) for holding a biological fluid, such as blood or a blood derivative, in which coagulation is measured. In this embodiment, a single magnetically susceptible particle (not shown) is disposed in each chamber. In other embodiments a small number of magnetically susceptible particles, for example 2 particles, or up to 10 particles are used per chamber. Two solenoids (108, 110) are positioned laterally of the test strip (102) and have arms (108a. 108b; 110a, 110b) extending from their cores (not shown) to distal ends close to the chambers (104, 106). In use, when one or other solenoid is supplied with direct current, the or each magnetically susceptible particle suspended in the biological fluid (not shown) traverse the chamber towards that solenoid. Then the respective other solenoid is energised to cause the or each particle to move back through the fluid, and the process repeated until coagulation occurs.

The or each chamber may be any convenient shape and its volume ranges typically from about 100 nL to 10 μL. The volume of blood or other fluid required by the device will depend upon the number of chambers and for a device having two chambers the volume requirement will typically range from about 250 nL-25 μL.

The detection chambers each have four unjacketed plastic 0.5 mm diameter fibre optics connecting the allowing the application of light by a respective light emitter (118a-d) and its detection by a respective optical detector (116a-d) over a restricted zone at the ends of the detection chambers, to optically interrogate the chamber. In the described embodiment each detector (116) is a respective photo-diode and each emitter is an LED (118) In another embodiment the emitter may be a laser diode.

As the or each magnetic particle traverses the chamber (104, 106) the detector/emitter pairs determine, by reflection of light from the lower surface of the chamber (104, 106) when, or whether, a particle is present in the region of the chamber (104, 106) covered by the detector-emitter (116, 118).

By switching the solenoids it is possible to determine, using the detector/emitter arrangement described above, when particles cease to traverse the chamber thereby indicating the coagulation of the biological fluid. It is alternatively possible to detect the transit time of the particles.

Referring to FIG. 2 an embodiment of the test strip (102) is formed of a lamina (103) of 125 μm thick PET coated on both sides coated on both sides with 25 g/m² pressure sensitive adhesive, and sandwiched by top and bottom laminae (described later herein). The lamina (103) has cut-outs forming part of the two chambers (104, 106) discussed above. The lamina (103) also has a sample application notch (2) for the biological fluid via a common inlet channel (3) to a bifurcation point (4). At bifurcation point (4) the common inlet channel (3) divides into two sample inlet channels (5, 6) serving the chambers (104, 106) respectively. In this embodiment, each of the chambers has dimensions of 2 mm×1 mm. Each of the chambers also has a respective vent channel (9, 10) connected to air exhausts (11, 12). The vent channels (9, 10) are partially cut channels which then become a fully cut wider channel at their distal ends This provides an effective capillary break to stop the egress of fluid from the test-strip (102).

In the embodiment shown typical volumes of the channels and notch are as follows:

Inlet notch 2=0.66 μl plus an open portion in layer 1 that if covered with blood gives a total for this region of approx 2.25 μl

Common inlet channel 3=0.71 μl

Sample Inlet channel 5=0.12 μl

Sample Inlet channel 60.42 μl

Vent channel 9=0.05 μl

Vent channel 10=0.05 μl

Chambers 104, 106, each have a volume of 350 nl.

The total internal volume is approximately 2.05 μl

In this particular embodiment, the inlet notch (2) has a volume of approx. 2.25 ul whereas the internal volume of the remaining part of the device is 2.05 ul. The inlet notch (2) is designed to fill with sample liquid and then supply that to the chambers, acting as a fill reservoir. The notch (2) enables a user to apply the sample from a source (for example from a pricked fingertip), and then remove the source without having to hold it there until the chambers have filled.

By contrast, without a notch of this type or a similar means of dispensing the liquid there could be a need for a user to maintain contact with a relatively hard-to-handle device, since to break contact could interrupt the flow of liquid, possibly leading to an air lock. This is especially advantageous for users of advanced years, or who have tremor or similar motor disorders.

In general providing a sample application reservoir of volume greater than the remaining internal volume of the device, liquid imparted into the reservoir is then able to fill the device. This is true providing the capillarity of the liquid conduit adjacent the liquid reservoir is greater than that of the reservoir such that liquid is automatically pulled into the device to empty the reservoir.

The features discussed above, defining the test strip are cut from the 125 μm thick PET. These features were cut using 2 passes of a laser using a 10 W CO₂ laser running at 70% power and 125 mm/s to minimise the amount of heat damage to material around the cut regions. However:—
The vent channels (9 and 10) were cut only once and so are effectively cut to depth. This minimises the volume of blood in the device and led to a depth change when sample reached the air exhaust creating an effective capillary break.
The common inlet channel (3) received 5 passes of the laser to ensure the cross sectional area was at least equivalent to the sum of the areas of the sample inlet channels (5 and 6). This pattern of laser cutting also helped to ensure a symmetric junction where the common channel splits.
The second sample inlet channel (6) received 3 passes of the laser such that it is cut to have an increased cross-sectional area with respect to the first sample inlet channel (5). As the fluid has further to travel this geometry reduces fluidic drag thereby allowing for the filling time of reaction chamber (104) to be substantially similar to that of reaction chamber (106).

An aspect of the invention provides for a method of creating microfluidic features by the use of a laser. In general a laser may be employed to cut a pattern into a substrate and a particular section of substrate subsequently removed to create a microfluidic feature such as a chamber. Alternatively, a microfluidic feature, such as a fluid conduit may be created by the cut-line of the laser itself. In the above example, a CO₂ laser was employed. Being of relatively low power, the CO₂ laser tends to melt the substrate thus creating the feature. A preferred alternative is to use a high power laser such as an excimer laser which tends to vaporise the substrate. Consequently much finer features may be obtained. Microfluidic structures obtainable using this method include fluid pathways, chambers, stepped fluidic elements. Regular spaced or irregularly spaced pillars may also be obtained by partially cutting down into the substrate at interval to ablate the material in between thus forming a protruding structure. The laser beam may be angled relative to the substrate to create angled walls and the fluid pathways may be straight or curved.

A cross-section (III-III′) of the lamina (103) is shown in FIG. 3. Release liners (301 and 305) cover the adhesive layers (302 and 304) over the lamina itself (303).

A thromboplastin coagulation reagent was then prepared from acetone dried brain powder (ADP). 2.5 g of ADP and 2.5 g Celite was mixed with 100 ml of a solution containing 0.85 g NaCl and 0.05 g deoxycholate for 30 min at 37° C. Following incubation the solution was centrifuged for 15 min at 1000 g at a temperature of 20° C. This supernatant residue was decanted and made up to 0.03% (v/v) phenol. The resulting solution was filtered by passing through filter paper and then made up to 3% (w/v) sucrose and 1% (v/v) ficol 70.

The thromboplastin solution was then placed in an airbrush reservoir and sprayed onto 100 μm thick clear PET film (403) using needle position setting 2.5 in areas to be bottom surfaces of sample chambers (104, 106).

Thromboplastin solution was sprayed using an EFD fluid handling system with the PET film placed on a XY platen moving at a rate of 30 mm/sec. The sprayed film was dried by heating to 45° C. for 10 min using an infrared lamp. These two layers were aligned such that the sprayed thromboplastin area was positioned under the reaction chambers. The sprayed film was aligned with the 125 μm PET film and the two layers were pressed together after removing the release liner (301, 305) from the 125 μm PET film.

FIG. 4 shows a cross-section taken along the line IV-IV′ of the lamina (103) adhered to the film (403). The view shows the laser cut chamber (104), prior to covering the device by adhering an upper lamina (not shown). The chamber (104) has a zone of thromboplastin (404) within the chamber (104). An aspect of the invention provides for a method of conveniently providing a reagent in a fluidic pathway, wherein the reagent is applied to a base substrate followed by lamination or folding of a further substrate or a further part of the base substrate onto the base substrate in order to define both the fluidic feature and the position of reagent relative to that feature. Deposition of the thromboplastin onto the substrate provides certain advantages over depositing the reagent into the chamber itself as it removes the need to have to accurately dose and position the reagent dispensing means. By providing the reagent initially on the lower substrate prior to assembly of a further laminate in order to define the reagent chamber, it may be provide for example as a striped band across a larger lower substrate. An upper laminate comprising a plurality of microfluidic features serving to define a plurality of individual test-strips may then be laminated onto the substrate comprising the reagent. The reagent may be positioned on the lower substrate such that after positioning of the upper laminate, the reagent is caused to be positioned within a chamber. Construction of the test-devices in this way removes the need to precisely locate the reagent as reagent which is positioned outside of the chamber will be effectively sandwiched between the two laminates and not form part of the microfluidic pathway. After assembling the individual laminate components in this way, individual test-strip may then be cut out which may be conveniently done by use of a laser.

Magnetic particles were prepared using 10 mg of iron spheres containing 0.5-5% silicon and a phosphatised surface (250-280 μm diameter) placed between two plates of Hi-speed (hardened) steel and then a pressure of 1000 psi was applied for 30 seconds. The resulting discs were sorted and those with a diameter between 400-600 μm and having a regular round shape were used for subsequent steps.

FIG. 5 shows a schematic diagram of the resulting discs (500). The discs have a diameter (501) of 400-600 μm and a thickness (502) of 70-80 μm.

Release liner (301) was removed and, in this embodiment, one disc (500) was placed in each reaction chamber (104, 106), close to the input port of the chamber.

Next as shown in FIG. 6, a section of 100 μm PET film (603) was placed such that a naturally hydrophilic surface faces the inside of the reaction chambers (104, 106). The test strip was then pressed to ensure all three plastic layers (103, 403, 603) adhere to each other.

The solenoid system is shaped to allow for a compact test device design, a shorter test-strip, a wider test-strip, a smaller blood volume as well as providing good proximity between the solenoid arms and the fluid chambers. The solenoids are also designed to minimise power consumption for a given magnetic field and to reduce power dissipation as heat. In the embodiment described, the solenoids dissipate less than 50 mW. A low heat dissipation is desirable so as not to interfere with the temperature of the test sample.

Each solenoid (700) has a single multi-turn winding (701), single core (not shown) and two arms (702, 703). This enables a close proximity of the arms to each chamber and only two solenoids (see FIG. 8). In this embodiment the arms (702, 703) have different lengths. This enables a shorter test-strip to be used. This in turn allows for a shorter fluid inlet passage and therefore a smaller blood volume. In other embodiments the arms may be of the same length.

An embodiment of the fluid chamber of FIG. 8 has the dimensions of 175 um in height×1000 um in width and a length of 2000 um which corresponds to a volume of 350 nL and which represents a ratio of the cross-sectional area of the particle to the cross-sectional area through which the particle moves of approximately 1:5. In this particular case, there are two chambers and an additional fluid conduit volume of 300 nL, thus requiring a total volume of 1 uL.

In the described embodiment, each solenoid arm (702, 703) is bifurcated at its distal ends to allow the test-strip to be slotted within the two forks. This allows a wider test-strip to be used providing strength and resilience to the test-strip yet allowing for a close proximity of the solenoid arms to the chamber. Due to the bifurcations it is also possible to create embodiments with chambers provided on the underside of the test-strip in a five layer laminated construct and that four chambers could be monitored simultaneously with only two solenoids. In an embodiment, the forks serve as a locating means for correctly positioning the test-strip in the test device. In embodiments the solenoid arms extend outwards from the main solenoid body such that the total length or width of the solenoid is greater than that of the main solenoid body itself. The solenoid arms may also have more than two forks.

Provision of a solenoid as described above having arms enables one solenoid to be used in place of two which results in a cost-saving as well as a reduction in overall size and weight of the reader.

Two solenoids (801, 802) are arranged around a test strip (102) as shown in FIG. 8.

The pulling force applied to a particle in the chamber (104, 106) by the magnetic field is proportional to the product of the magnetic field strength and the magnetic field gradient. The geometry of the solenoid arms is designed to give a magnetic field shape that pulls the particle across the measurement chamber. The geometry is a combination of both solenoids, the particles in their measurement chambers and the relative spacing between them. Each solenoid is turned on at time-spaced intervals, and the magnetic flux generated by the energized solenoids passes between its solenoids arm tips. The relatively high magnetic permeability path through the particle and the arms and core of the non-energised coil attracts a proportion of this flux. This gives the magnetic field the shape that allows it to pull the particle across the chamber.

A solenoid drive circuit drive the solenoids according to the timing intervals as shown in FIG. 9.

This cycle is arranged so the switching of the two solenoids (801, 802) runs at a 500 ms timing cycle. The cycle starts at 0 ms (903) when first solenoid (801) is activated. The coils are driven with the battery voltage that is switched across the solenoid at a frequency of 5 kHz and pulse width modulated to allow for variations in battery voltage. The switched current is self-smoothed by the resistance and inductance of the coil to give a DC current equivalent to that a 1.5V supply would give if applied continuously to the coil. After 100 ms (904) first solenoid (801) is turned off. At 250 ms into the cycle (905), second solenoid (802) is activated using the same driving conditions as used on solenoid 1. After 350 ms into the cycle (906) this solenoid is switched off. At 500 ms the cycle repeats (907).

A drive circuit illuminates the LEDs (118) and a detector circuit detects signals from the detectors (116) according to the timing intervals as shown in FIG. 10. This cycle is arranged so the switching of the four LEDs (118) run as a 500 ms timing cycle which is synchronised to the solenoid drive waveform. The cycle starts at 0 ms (915) with a first LED (118a) for chamber (106) already switched on. Just before this LED is switched off 100 ms into the cycle (916) the signal from the corresponding detector (116a) from the optical fibre is measured. At 100 ms the second LED (118b) for chamber (106) is switched on. 150 ms into the cycle (917) just before this LED is switched off the signal from the corresponding detector (116b) from the optical fibre is measured. At 150 ms the LED (118c) for chamber (104) is switched on and just before this is switched off at 200 ms (918) the output from the corresponding detector (116c) from the optical fibre is measured. At 200 ms (918) the other LED (118d) for chamber (104) is switched on. At 250 ms (919) the output from the corresponding detector (116d) from the optical fibre is measured. This LED is illuminated until 350 ms into the cycle (920) and just before it is switched off the output from the detector is measured a second time. At 350 ms into the cycle (920) the other LED (118c) for chamber (104) is illuminated. Just before this is switched off at 400 ms into the cycle (921) the output from the corresponding detector (116d) from the optical fibre is measured. At 400 ms (921) the second LED (118b) for chamber (106) is switched on. Just before this is switched off at 450 ms into the cycle (922) the output from the detector (116a) from the optical fibre is measured. At 450 ms into the cycle the other first LED (118a) for chamber (106) is illuminated and at the end of the cycle at 500 ms (923) the output from the detector from the optical fibre is measured. The switching cycle then continues to repeat. The detectors are electronically connected such that the outputs from these generate output in a single channel. Synchronisation of the magnetic wave form with the optical interrogation means in an offset way enables a single signal processing means to be employed for all measurements. Consequently this reduces the amount of electronic components which in turn reduces cost and overall size of the reader.

There is an advantage of having two pairs of fibre optics located around each detection chamber. Blood entering the chamber at one end can be detected from one pair of fibre optics and blood filling the chamber can be detected through the second pair of fibre optics in the chamber. This allows the timing of blood entry and blood fill to be identified.

As will be appreciated from the above description, two measurements from a detection window are taken within one cycle, one when the particle is (or should be) not present in the detection window and one when the particle is (or should be) present in the detection window. Using this data it is possible to determine the position of the particle within the chamber. The use of 2 fibre optic pairs across a single chamber enables any particle that stops or is momentarily held up on the edge of one of the fields of view to be detected. In this way it is possible to determine relative changes in optical signal due to the movement of the particle.

A test strip placed between the solenoids with an optical assembly interrogating the chambers was used to detect a clotting event in whole blood. A finger stick blood sample was applied to the end of the device. The signal output when each of the four LEDs are illuminated is shown in FIG. 11. Blood can be seen entering (1001) and filling (1002) the first chamber and then entering (1003) and filling (1004) the second chamber. The clotting of blood can then be seen in both chambers (1005, 1006).

Embodiments of the device advantageously have a total volume of chambers plus filling channels of less than or equal to 3 μl. A device having a volume of 2 μl can be derived from the FIG. 1 embodiment. By combining sizes from the FIGS. 1 and 8 embodiments, volumes of 1.5 μl, 1 μl and 350 nl may be achieved. Where very small volumes are desired, say down to 250 nl or even to 100 nl, special measures may be needed. An exemplary device of such very low volume has the needle used for penetration of the skin integral with the test strip to reduce transfer losses. In this situation the needle or lancet may incorporate microfluidic channels to allow for automatic transfer of blood into the chamber.

An embodiment of the invention has now been described. The invention itself is not restricted to the described features but instead extends to the full scope of the appended claims.

1. A device for use with a reader for determining coagulation of a sample of biological fluid, the device comprising a structure having a chamber for containing a sample of biological fluid, and wherein a coagulation reagent capable of interacting with the fluid sample is provided within the device, the chamber containing a number of particles susceptible to movement in a magnetic field.

2. A device according to claim 1, wherein each particle has a major axis greater than 5 um in length, more particularly a major axis between 5 um and 12 um in length, and more particularly still a major axis that is substantially 10 um in length.

3. A device for use with a reader for determining coagulation of a sample of biological fluid, the device comprising a structure having at least one chamber for containing a sample of biological fluid, and wherein a coagulation reagent capable of interacting with the fluid sample is provided within the device, the at least one chamber containing one particle susceptible to movement in a magnetic field.

4. A device according to claim 3, wherein the particle has a major axis between 300 um and 700 um in length, more particularly a major axis between 400 um and 600 um in length, and more particularly still a major axis that is substantially 500 um in length, more particularly a thickness between 50 um and 100 um, and more particularly still a thickness that is substantially 70 um.

5. A device according to claim 3, wherein the particle is shaped like one of the groups of shapes comprising: a disc, a sphere, a torus, an ellipsoid, and an oblate spheroid.

6. A device according to claim 1, wherein the chamber contains between two and ten particles.

7. A device according to claim 1, wherein each particle is initially disposed towards one of an inlet or outlet port.

8. A device according to claim 1, wherein each particles is disc shaped.

9. A device according to claim 1, wherein said structure is formed of a plurality of laminae.

10. A device according to claim 9, wherein one of said lamina defines a geometry of said chamber.

11. A device according to claim 1, further comprising passages for introduction into the chamber of said fluid.

12. A device according to claim 1 having two chambers.

13. A reader for use with the device of claim 1, the reader comprising:—
magnetic means arranged to successively apply first and second magnetic fields to cause a said particle to move to and fro within the chamber; and
optical monitor means associated with the chamber to establish a change in said to and fro particle movement.

14. A reader according to claim 13 wherein said optical monitor means comprises one or a plurality of emitter/detector pairs.

15. A reader according to claim 13, wherein respective optical monitoring means are disposed to monitor respective ends of said chamber.

16. A reader according to claim 14, wherein each of said emitters and detectors are optically coupled to said chamber by optical wave-guides.

17. A reader according to claim 13, wherein said magnetic means comprises at least one solenoid, preferably having a winding, a core and two arms extending from the core defining a portion of a magnetic circuit.

18. A reader according to claim 17, wherein said two arms have different lengths.

19. A system for determining coagulation of a sample of biological fluid, comprising a magnetic drive means and structure defining a chamber, the chamber containing a particle capable of moving under the influence of a magnetic field, the magnetic drive means being arranged in use to co-operate with the particle to cause it to move back and forth in the chamber, the device further comprising at least one light detection means having an input disposed to be selectively occluded by the said particle.

20. A method of determining the coagulation status of a sample of biological fluid by interaction with a coagulation reagent, the method comprising, in any order of steps (a)-(c) or simultaneously:
(a) causing the sample of biological fluid to become disposed in a device, the device having a chamber containing a particle which is susceptible to movement in a magnetic field;
(b) successively applying first and second magnetic fields to cause the said particle to move to and fro within the chamber;
(c) optically monitoring the chamber to establish a change in said to and fro movement of said particle and:
(d) correlating the change in particle movement with the coagulation status of the fluid sample.

21. A method according to claim 20, wherein the coagulation reagent is disposed in the device prior to step a).

22. A method according to claim 20, wherein said particle is an iron sphere.

23. A method according to claim 22, wherein said iron spheres contain silica.

24. A device for use with an optical reader for determining coagulation of a sample of biological fluid, having a chamber for containing a said sample and a channel for admitting said biological fluid into said chamber, wherein the channel and the chamber together have a volume of less than 3 μL.

25. A device according to claim 24 wherein the volume is less than 1 uL.

26. A device according to claim 24, wherein the volume is less than 250 nL.

27. A device according to claim 24, wherein the volume is substantially 100 nL.

28. A device according to claim 24, having integral means for penetrating the skin, the said means defining a conduit which forms at least part of said channel.

29. A device according to claim 3, wherein the particle is initially disposed towards one of an inlet or outlet port.

30. A device according to claim 3, wherein the particle is disc shaped.

31. A device according to claim 3, wherein said structure is formed of a plurality of laminae.

32. A device according to claim 31, wherein one of said lamina defines a geometry of said chamber.

33. A device according to claim 3, further comprising passages for introduction into the chamber of said fluid.

34. A device according to claim 3, having two chambers.

35. A device according to claim 3, wherein the particle is a disc shaped pressed iron sphere.

36. A device according to claim 1, wherein at least one particle is a disc shaped pressed iron sphere.

37. A system according to claim 19, wherein the particle is a disc shaped pressed iron sphere.
